(19) United States
(12) Patent Application Publication
(10) Pub. No.: US 2009/0289015 A1
(43) Pub. Date: Nov. 26, 2009

(54) BACK-SURFACE MIRRORS FOR ULTRAVIOLET LIQUID DISINFECTION SYSTEMS

(76) Inventor: Uri LEVY, Rehovot (IL)

Correspondence Address: Pearl Cohen Zedek Latzer, LLP, 1500 Broadway, 12th Floor, New York, NY, 10036, US

(21) Appl. No.: 12/124,190

(22) Filed: May 21, 2008

(51) Int. Cl. A61L 2/10; B01J 19/12; C02F 1/32

(52) U.S. Cl. 210/748; 422/186.3; 210/198.1

(57) ABSTRACT

According to embodiments of the invention, ultraviolet-based liquid disinfection systems with back-surface mirrors for enhancing the performance are provided. The disinfection system may include, for example, metallic mirrors deposited externally over areas of a UV-transparent liquid conduit to redirect light rays back into the conduit

### BACKGROUND

Ultraviolet (UV) liquid disinfection systems using UV light source located within a metallic chamber through which the liquid flow have been long known. The UV source is enclosed in a quartz sleeve immersed in the liquid to be treated and the UV light propagates through the liquid. The walls of the metallic chamber, however, absorb most of the incident UV light and accordingly light rays emitted from the UV light source traverse through the liquid only before being absorbed by the metal. Therefore, the UV light is not being utilized in an efficient manner.

The irradiation of the liquid inactivates microorganisms in the liquid, if the irradiation intensity and exposure duration are above a minimum dose level (often measured in units of miliJoules per square centimeter). Further, higher irradiation intensity would cause higher level of inactivation of the microorganisms within the chamber. Ideally, UV-based disinfection systems should be constructed such that each microbe that crosses the chamber in any possible stream line is irradiated with the same UV dose.

There is a need for a UV disinfection system that would be more efficient than existing systems.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is an illustration of a disinfection system according to some demonstrative embodiments of the invention;

FIG. 2 is an illustration of a disinfection system according to some demonstrative embodiments of the invention; and

FIG. 3 is an illustration of a disinfection system with an external UV light source according to some demonstrative embodiments of the invention.

It will be appreciated that for simplicity and clarity of illustration, elements shown in the drawings have not necessarily been drawn accurately or to scale. For example, the dimensions of some of the elements may be exaggerated relative to other elements for clarity or several physical components included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION OF EMBODIMENTS OF THE INVENTION

In the following detailed description, numerous specific details are set forth in order to provide a thorough understanding of the invention. However, it will be understood by those of ordinary skill in the art that the present invention may be practiced without these specific details. In other instances, well-known methods, procedures, components and circuits may not have been described in detail so as not to obscure the present invention.

Embodiments of the present invention are directed to an ultraviolet (UV) disinfection system for treating liquid flowing in a conduit coated with a UV reflective coating on its exterior surfaces. The reflective surface may produce a rear surface mirror effect, thus increasing the efficiency of the system by allowing light emitted from a UV light source to reflect back into the liquid.

It will be appreciated that the liquid disinfection process may include inactivation or removal of any organism, bacteria, microorganism, being, creature, microbe, germ, virus, organic contaminator, non-organic contaminator, oxidizeable toxic or contaminator; any cumulative noxious species of biological or chemical origin; any oxidizing particle, fragment or element, e.g., Hydrogen peroxide or Titanium dioxide, intended to oxidize a contaminator and/or the like.

In some demonstrative embodiments of the invention, the device may include a conduit, for example, a reactor, a vessel, a chamber, e.g., an elongated chamber, to carry the liquid. The conduit may have an inlet to receive the liquid and an outlet to discharge the liquid. The system may also include at least one illumination source, such as Ultraviolet (UV) source to illuminate the conduit with light. Some demonstrative embodiments of the invention may refer to using UV light to disinfect the liquid and/or to oxidize the particles with the liquid. However, it will be appreciated by those skilled in the art, that in other embodiments of the invention, light of any other suitable spectrum may be used.

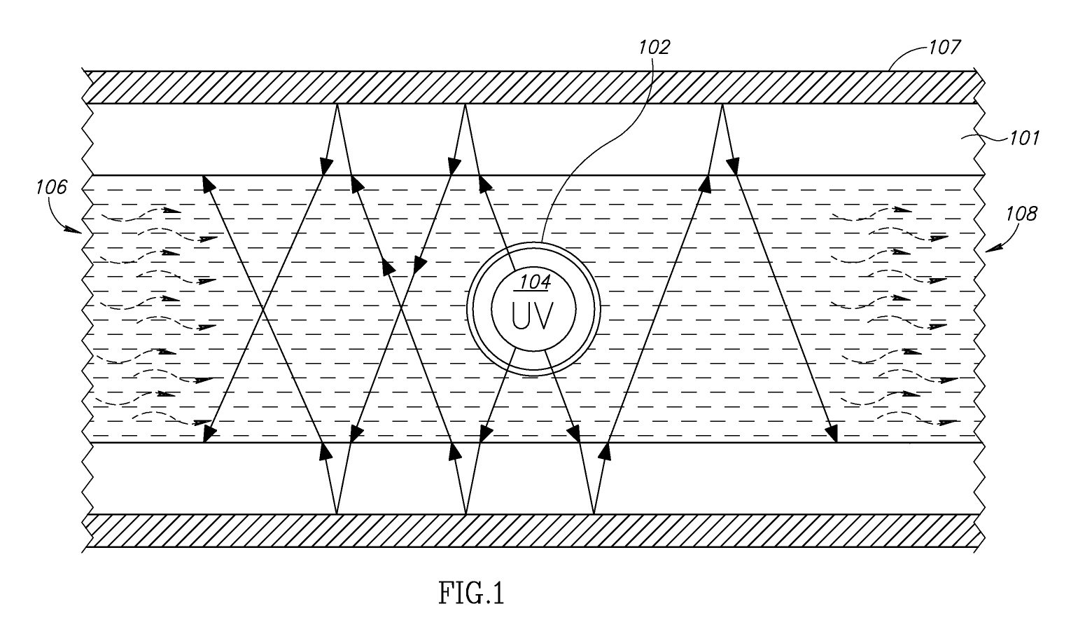

Reference is now made to FIG. 1, which conceptually illustrates a disinfection system according to some demonstrative embodiments of the invention. A disinfection system may include a tube or conduit 101 to carry liquid to be disinfected, one or more substantially light-transparent sleeves 102 positioned within conduit 101 substantially parallel to its longitudinal axis of symmetry and one or more light sources 104, each positioned within a respective sleeve 102. Illumination sources 104 may be UV light sources capable of emitting light at 254 nm. Conduit 101 may have an inlet 106 to receive, for example, from an external liquid pipe the liquid to be disinfected and an outlet 108 to discharge the liquid via an external discharge pipe. The system may further include adaptors (not shown) to connect conduit 101 to the external liquid pipes.

FIG. 1 illustrates for simplicity the use of a single UV source. It should, however, be understood to a person skilled in the art that embodiments of the invention are not limited in this respect and any number of UV sources is likewise applicable. It should also be noted that the UV source is not bound by any specific location, rather the UV source may be located anywhere within the conduit.

Although the invention is not limited in this respect, the UV source may be parallel to the longitudinal axis of symmetry of the conduit. It should, however, be understood to a person skilled in the art that embodiments of the invention are not limited in this respect and the UV source may set at any angle with respect to the longitudinal axis of symmetry of the conduit.

Although the invention is not limited in this respect, the illumination source may generate UV light of a suitable germicidal UV spectrum. For example, illumination source may include one or more UV lamps, e.g., a low-pressure UV, a medium-pressure UV lamp and/or a microwave-excited UV lamp, as are all known in the art.

Conduit 101 may be substantially made of UV-transparent glass, such as quartz. UV-transparent sleeves 102 may be for example quartz or Teflon® sleeves. Illumination sources 104 may illuminate the liquid to be disinfected when flowing in the conduit. Conduit 101 may be located inside a protective metal sleeve with an air gap between the conduit and the sleeve (not shown).

According to some embodiments of the present invention, substantially all of the exterior surface of conduit 101 may be coated with UV reflective coating 107 to produce rear surface mirror effect, e.g., to allow a larger portion of the light from illumination source 104 to illuminate the liquid flowing in conduit 101. According to other embodiments of the invention only a portion of the exterior surface of conduit 101 may be coated with UV reflective coating 107. Coating 107 may reflect back into the liquid additional light rays reaching the surface in relative proximity to sleeve 102.

Reflective coating 107 may comprise aluminum deposition, gold deposition or multi-layer dielectric material. Any other suitable reflective coating may be used. As can be seen in FIG. 1, light emitted from light source 102 may reach the internal surface of conduit 101, traverse the transparent wall to be reflected back into the liquid by the UV reflective coating. Once the light is reflected back into the liquid, the light may then traverse a second transparent wall, and then once again be reflected back into the liquid. This process may be repeated as long as there is sufficient energy present in the light.

According to some embodiments, the light emitted from light source 102 may hit reflective coating 107 at any angle. It is not important that the emitted light be at a certain angle, as substantially all light that reaches reflective coating 107 may be reflected back into conduit 101.

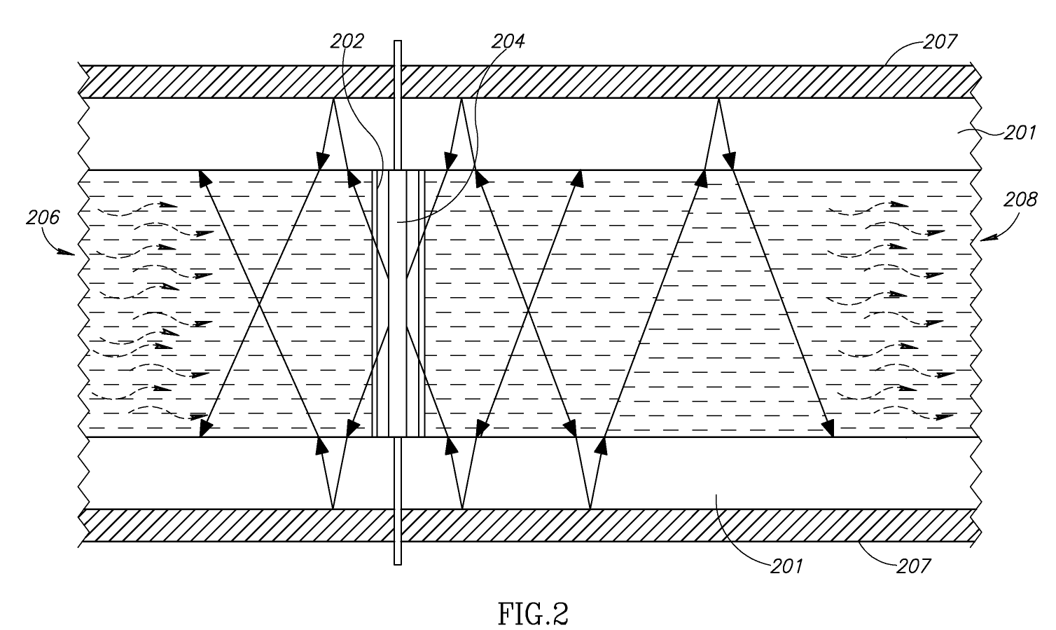

Reference is now made to FIG. 2, which conceptually illustrates a disinfection system according to some demonstrative embodiments of the invention. A disinfection system may include a tube or conduit 201 to carry liquid to be disinfected, one or more substantially light-transparent sleeves 202 positioned within conduit 201 substantially perpendicular to its longitudinal axis of symmetry and one or more illumination sources 204, each positioned within a respective sleeve 202. According to embodiments of the invention, light sources 202 may be UV light sources capable of emitting light at 254 nm. Conduit 201 may have an inlet 206 to receive from an external liquid pipe the liquid to be disinfected and an outlet 207 to discharge the liquid via an external discharge pipe. The disinfection system may further include adaptors (not shown) to connect conduit 201 to the external liquid pipes.

Conduit 201 may be substantially made of UV-transparent glass, such as quartz. UV-transparent sleeves 202 may be for example quartz or Teflon® sleeves. Each Sleeve 202 may have external dimensions smaller than the internal dimensions of conduit 201 such that liquid may flow within conduit 201 around sleeves 202. Both ends of sleeve 202 may extend from the walls of conduit to enable replacement of light source 204 within sleeve 202. Illumination sources 204 may illuminate the liquid to be disinfected when flowing in the conduit. Conduit 201 may be located inside a protective metal sleeve with an air gap between the conduit and the sleeve (not shown).

According to some embodiments of the present invention, substantially all of the exterior surface of conduit 201 may be coated with UV reflective coating 207 to produce rear surface mirror effect, e.g., to allow a larger portion of the light from illumination source 204 to illuminate the liquid flowing in conduit 201. According to other embodiments of the invention only a portion of the exterior surface of conduit 201 may be coated with UV reflective coating 207. Coating 207 may reflect back into the liquid additional light rays reaching the surface in relative proximity to sleeve 202.

Reflective coating 207 may comprise aluminum deposition, gold deposition or multi-layer dielectric material. Any other suitable reflective coating may be used. As can be seen in FIG. 2, light emitted from light source 202 may reach the internal surface of conduit 201, traverse the transparent wall to be reflected back into the liquid by the UV reflective coating.

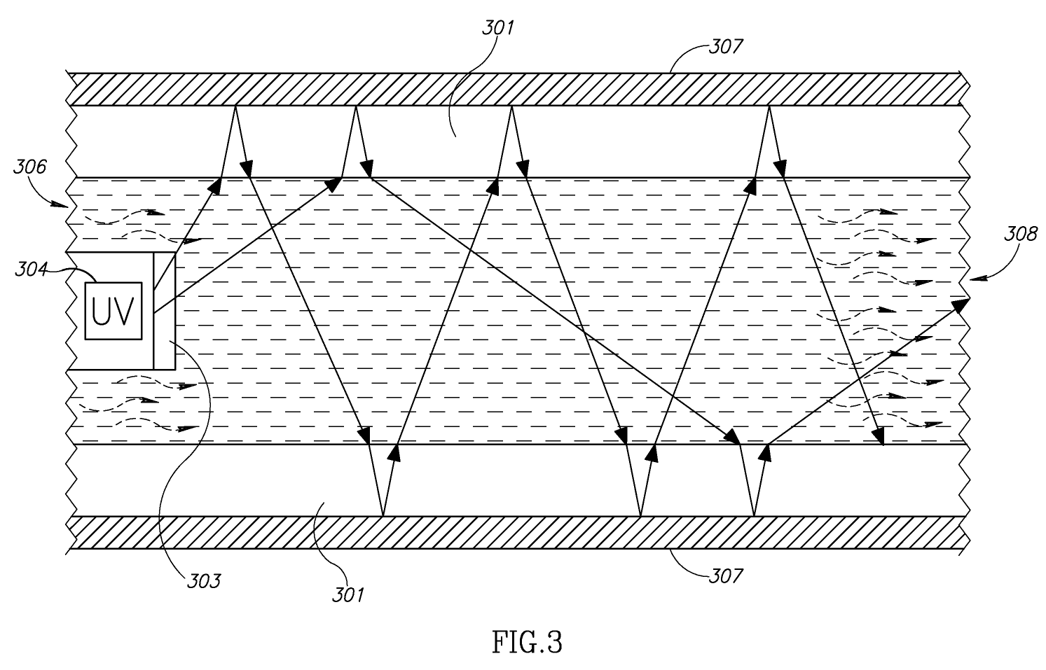

Reference is now made to FIG. 3, which conceptually illustrates a disinfection system according to some demonstrative embodiments of the invention. A disinfection system may include a tube or conduit 301 to carry liquid to be disinfected and an external illumination source 304 to illuminate the liquid within conduit 301. Illumination sources 304 may be UV light sources capable of emitting light at 254 nm. Conduit 301 may have an inlet 306 to receive, for example, from an external liquid pipe the liquid to be disinfected, and an outlet 308 to discharge the liquid via an external discharge pipe. The system may further include adaptors (not shown) to connect conduit 101 to the external liquid pipes.

Conduit 301 may be made of UV-transparent material, such as quartz, and a transparent window 303 located at one end of conduit 301 proximate to illumination source 304. Any other suitable light-transparent material may be used. The light produced by illumination source 304 may be directed toward the liquid within conduit 301 via window 303. The conduit may be located inside a protective metal sleeve with an air gap between the conduit and the sleeve (not shown).

According to some embodiments of the present invention, substantially all of the exterior surface of conduit 301 may be coated with UV reflective coating 307 to produce rear surface mirror effect, e.g., to allow all of the light from illumination source 304 to illuminate the liquid flowing in conduit 301. According to other embodiments of the invention only a portion of the exterior surface of conduit 301 may be coated with UV reflective coating 307. Coating 307 may reflect back into the liquid additional light rays reaching the surface in relative proximity to window 303.

Reflective coating 307 may comprise aluminum deposition, gold deposition or multi-layer dielectric material. Any other suitable reflective coating may be used. As can be seen in FIG. 3, light emitted from illumination source 304 may reach the internal surface of conduit 301, traverse the transparent wall to be reflected back into the liquid by the UV reflective coating.

According to some embodiments of the invention, a reflector (not shown) may partially surround illumination source 304. The reflector may be positioned such that radiation from illumination source 304 may be reflected from the reflector in the direction of window 303. The reflector is designed to deflect light into conduit 301 through window 303 in a way that maximizes the UV output from illumination source 304.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

1. An apparatus for liquid disinfection by ultraviolet (UV) light, the apparatus comprising:
a conduit to carry a flowing liquid to be disinfected, said conduit having UV-transparent walls, an inlet to receive said liquid and an outlet to discharge said liquid, wherein an external surface of said UV-transparent walls is coated with a UV reflective coating; and
at least one UV source to illuminate said liquid with UV light, wherein the UV source is positioned within the conduit substantially perpendicular to a longitudinal axis of symmetry of the conduit and to the liquid flow direction such that a light ray emitted from the UV source is reflected by the reflective coating into the liquid more than once while advancing away from the UV source.

2. The apparatus of claim 1, wherein the UV reflective coating produces a back-surface mirror effect such that light emitted from the UV source reach an internal surface of the walls, traverse the walls and reflect back into the liquid.

3. The apparatus of claim 1, wherein the UV reflective coating comprises aluminum, gold or multi-layer dielectric material.

4. The apparatus of claim 1, wherein the conduit comprises quartz.

5-8. (canceled)

9. A method for liquid disinfection by ultraviolet (UV) light comprising:
flowing liquid to be disinfected through a conduit, said conduit having UV-transparent walls, wherein an external surface of said UV-transparent walls is coated with a UV reflective coating;
illuminating said flowing liquid with UV light emitted from a UV source positioned within the conduit substantially perpendicular to a longitudinal axis of symmetry of the conduit and to the liquid flow direction; and
reflecting said UV light from the reflective coating such that a light ray emitted from the UV source is reflected by the reflective coating into the liquid more than once while advancing away from the UV source.

10. The method of claim 9, wherein illuminating the flowing liquid comprises emitting the UV light from the UV source such that at least a portion of the UV light reach an internal surface of the walls at least twice, traverse the walls and reflect back into the liquid

11-14. (canceled)
